(19) United States
(12) Patent Application Publication
(10) Pub. No.: US 2008/0289248 A1
(43) Pub. Date: Nov. 27, 2008

(54) IMMOBILIZED ESTERIFICATION CATALYSTS FOR PRODUCING FATTY ACID ALKYL ESTERS

(75) Inventor: Yong Gao, Carbondale, IL (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: Southern Illinois University Carbondale, Carbondale, IL (US)

(21) Appl. No.: 11/752,666

(22) Filed: May 23, 2007

(51) Int. Cl. C10L 1/19; C07C 67/08

(52) U.S. Cl. 44/308; 554/159

(57) ABSTRACT

Provided herein are processes for the production of biodiesel. In particular, provided is an esterification process in which an alcohol reacts with free fatty acids in a lipid material comprising free fatty acids and glycerides in the presence of an immobilized zirconium(IV) metal salt to form fatty acid alkyl esters. Also provided is combination process in which an esterification reaction converts the free fatty acids in a lipid material to fatty acid alkyl esters and a transesterification reaction converts the glycerides in the material to fatty acid alkyl esters.

### GOVERNMENTAL RIGHTS

The present invention was supported by funding from the National Science Foundation through a Faculty Early Career Development Award (CHE-0343440) and the Small Business Technology Transfer Program (STTR) (IIP-071 1652) and the National Institutes of Health (1R15EB007074-01). The United States Government has certain rights in this invention.

### FIELD OF THE INVENTION

The present invention provides processes for forming fatty acid alkyl esters in a lipid material comprising free fatty acids and glycerides.

### BACKGROUND OF THE INVENTION

Biodiesel is an alternate fuel produced from natural, renewable biological materials. Typically, biodiesel comprises fatty acid methyl esters produced through a transesterification reaction between the triglycerides in vegetable oils or animal fats with methanol in the presence of a transesterification catalyst, such as sodium hydroxide. Currently, most biodiesel is made from vegetable oils, but there are large amounts of fats and oils unsuitable for human consumption that could be converted to biodiesel at lower cost. The problem with these alternate feedstocks, however, is that they often contain large amounts of free fatty acids that cannot be converted to biodiesel using an alkaline catalyst. These free fatty acids react with the alkaline catalyst to produce soaps that inhibit the separation of the biodiesel from the other byproducts. Removal of the free fatty acids from the feedstocks not only lowers the biodiesel yields, but also increases the overall manufacturing costs due to additional separation and/or purification steps.

Attempts have been made to identify esterification catalysts that would convert the free fatty acids into fatty acid alkyl esters. As an example, sulfuric acid is generally the catalyst of choice for converting free fatty acids in soapstocks. Sulfuric acid, however, is corrosive, and the removal of sulfuric acid after the esterification reaction requires the use of expensive equipment. Additionally, the use of sulfuric acid frequently leads to high sulfur levels in the final biodiesel product.

In recent years, some multivalent metal salts such as ZrOCl₂.8H₂O, HfOCl₂.8H₂O, and ZnCl₂ were found to be effective catalysts in esterification reactions (Ishihara et al., Tetrahedron 2002, 58, 8179-8188; Mantri et al., Green Chemistry, 2005, 7, 677-682). Many of these Zr(IV) and Hf(IV) salts, however, are highly soluble in polar solvents (e.g., methanol) at the temperature of the reaction (typically greater than about 100° C.), which limits the recovery of the catalyst. Measures devised to address this problem included using nonpolar organic solvents (such as heptane, in which metal salts are less soluble), cooling the reaction products after the reaction to precipitate the catalyst, or using an N-(polystyrylbutyl)pyridinium polymer to capture the metal oxide catalyst (Nakayama et al., Adv. Synth. Catal. 2004, 346, 1275-1279; Nakamura et al., Adv. Synth. Catal. 2006, 348, 1505-1510). None of these remedies is ideal, however. Using a metal salt catalyst in a polar reagent like methanol, for example, increases the likelihood of metal contamination in the final biodiesel product. And introducing a second less polar solvent like hexane increases the cost of production in terms of raw materials and waste control.

Thus, there is still a need for efficient esterification catalysts that would quickly and cost effectively convert fatty acids in a feedstock to fatty acid alkyl esters. Ideally, the catalyst should be recoverable and recyclable. Further, it is envisioned that the esterified feedstock would be subjected to a transesterification reaction to form biodiesel. What is needed, therefore, is a continuous process (e.g., employing a fixed bed reactor) for the esterification of free fatty acids and the subsequent transesterification of triglycerides to produce biodiesel from a variety of feed stocks.

### SUMMARY OF THE INVENTION

One aspect of the present invention provides a process for forming fatty acid alkyl esters. The process comprises contacting a lipid material with an alcohol in the presence of a zirconium(IV) metal salt conjugated to a solid support at a temperature of about 25° C. to about 75° C. During this reaction, the alcohol reacts with free fatty acids in the lipid material to form fatty acid alkyl esters.

Another aspect of the invention provides a process for forming a biodiesel solution. The process comprises sequential esterification and transesterification reactions that convert the free fatty acids and the glycerides in a lipid material to fatty acid alkyl esters. The process comprises contacting the lipid material with an alcohol in the presence of a zirconium(IV) metal salt catalyst, during which the free fatty acids are esterified. The process further comprises contacting the esterification reaction mixture with a transesterification catalyst (i.e., either an N-heterocyclic carbene or an alkaline catalyst). The transesterification reaction converts the glycerides to fatty acid alkyl esters, thereby forming the biodiesel solution.

Other aspects of the invention are described in more detail herein.

### DETAILED DESCRIPTION OF THE INVENTION

The present invention provides processes for forming fatty acid alkyl esters or “biodiesel” from feedstocks comprising free fatty acids and glycerides. It has been discovered that fatty acid alkyl esters may be formed through an esterification reaction between a free fatty acid and a short chain alcohol in the presence of an immobilized zirconium(IV) metal salt catalyst at a temperature of less than about 75° C. Immobilization of the catalyst by conjugation to a solid support enables the catalyst to be readily recovered and reused, thereby reducing the cost of biodiesel production. Furthermore, esterification of the free fatty acids in a feedstock eliminates the need to remove the free fatty acids prior to a transesterification reaction, thereby further reducing the cost of biodiesel production. Thus, the invention provides a combination esterification/transesterification process for forming biodiesel from, preferably, cheap feedstocks that contain significant amounts of free fatty acids.

### (I) An Esterification Process for Forming a Fatty Acid Alkyl Ester

One aspect of the present invention provides a process for reacting a free fatty acid with an alcohol to form a fatty acid alkyl ester. The process comprises contacting a lipid material with an alcohol in the presence of a zirconium(IV) metal salt conjugated to a solid support at a temperature from about 25° C. to about 75° C. The immobilized zirconium(IV) metal salt catalyst may be readily recovered after the reaction, recycled, and reused.

As used herein, an esterification reaction is the chemical process of condensing a fatty acid with an alcohol in the presence of a catalyst. The products of the reaction are a fatty acid ester and water.

The fatty acid esters formed by processes of the invention may be used as fuel compositions (e.g., biodiesel), lubricants, emulsifiers, plasticizers, or in a variety of other applications.

(a) Lipid Material

The lipid material comprises free fatty acids and glycerides and is generally derived from a renewable material. The renewable material may be a vegetable oil, an animal fat, an algae oil, a food-based lipid waste, an industrial lipid waste, or a combination thereof. Non-limiting examples of suitable vegetable oils include artichoke oil, camelina oil, canola oil, castor oil, coconut oil, copra oil, corn oil, cottonseed oil, flaxseed oil, hemp oil, jatropha oil, jojoba oil, karanj oil, milk brush/pencil bush oil, mustard seed oil, neem oil, olive oil, palm oil, peanut oil, radish oil, rapeseed oil, rice bran oil, rubber seed oil, safflower oil, sesame oil, soybean oil, sunflower oil, and tung oil. Suitable animal fats include, but are not limited to, blubber, chicken fat, cod liver oil, fish oil, ghee, poultry fat, lard, and tallow. Suitable fish oils include anchovy oil, herring oil, lake trout oil, mackerel oil, menhaden oil, pollock oil, salmon oil, and sardine oil. Non-limiting examples of algae oils include those from Aphanizomenon flosaquae, Bacilliarophy sp., Botryococcus braunii, Chlorophyceae sp., Crypthecodinium cohnii, Dunaliella tertiolecta, Euglena gracilis, Isochrysis galbana, Nannochloropsis salina, Nannochloris sp., Neochloris oleoabundans, Phaeodactylum tricornutum, Pleuro chfysis carterae, Prymnesium parvum, Scenedesmus dimorphus, Schizochytrium sp., Spirulina sp., and Tetraselmis chui. Examples of food-based lipid waste include waste vegetable oil (WVO), spent frying oil, yellow grease, which is the reusable grease obtained from restaurant operations, and brown grease, which is the grease collected via the wastewater stream through a passive trap or interceptor. Suitable examples of industrial lipid waste include deodorizer distillates and acid oils (soapstocks) generated as side streams during the production of oil and detergent products; tall oils, which are byproducts of the pulping of pinewood; and red oils from the candle industry. As will be appreciated by the skilled artisan, the lipid material may be a combination of materials derived from different sources. For example, the lipid material may be a combination of a vegetable oil and an animal fat

The lipid material generally comprises a mixture of free fatty acids and glycerides. One skilled in the art will appreciate that different lipid materials have different proportions of free fatty acids and glycerides. For example, crude soybean oil typically comprises about 5% of free fatty acids, yellow grease may comprise about 15% of free fatty acids, and brown grease may comprise about 40% of free fatty acids.

The lipid material may be used without any pretreatment, or the lipid material may be pretreated prior to contact with the alcohol and the catalyst. The source of the lipid material and the desired degree of purity of the fatty acid ester products will generally determine whether the lipid material is pretreated. One process of pretreatment comprises degumming to remove phosphatides, gums, other solids, and metals from the lipid material. Another pretreatment process comprises vacuum drying such that the water content of the lipid material is reduced to about 10% or less by weight of the lipid material. Other pretreatment processes comprise steam distillation and/or bleaching to remove protein, volatile impurities, color pigments, and/or other impurities from the lipid material. The above-mentioned processes, as well as other pretreatment methods, are well known in the art.

(b) Alcohol

In general, any alcohol may be used in the esterification process of the invention. An alcohol comprises any compound having at least one hydroxyl group bound to a carbon atom of an alkyl or a substituted alkyl group. Thus, an alcohol may be linear, cyclic, or branched, and the hydrocarbyl moiety may be saturated or unsaturated. Alcohols suitable for use in this invention will generally have less than about 10 carbon atoms. In one embodiment, the alcohol may have from about 8 carbon atoms to about 10 carbon atoms. In another embodiment, the alcohol may have from about 5 carbon atoms to about 7 carbon atoms. In a preferred embodiment, the alcohol may have from 1 carbon atom to about 4 carbon atoms. Suitable alcohols having from 1 carbon atom to about 4 carbon atoms include methanol, ethanol, propanol, isopropanol, butanol, and isobutanol. In an exemplary embodiment, the alcohol may be methanol. It should be noted that combinations of alcohols may also be used in the process of the invention.

The concentration of alcohol used in the esterification reaction can and will vary depending upon a variety of factors, including the source of the lipid material. The concentration of alcohol may range from about 1 % to about 2000% by weight of the lipid material. In one embodiment, the concentration of alcohol may range from about 1% to about 50% by weight of the lipid material. In another embodiment, the concentration of alcohol may range from about 50% to about 100% by weight of the lipid material. In an alternate embodiment, the concentration of alcohol may range from about 100% to about 500% by weight of the lipid material. In still another embodiment, the concentration of alcohol may range from about 500% to about 1000% by weight of the lipid material. In yet another embodiment, the concentration of alcohol may range from about 1000% to about 1500% by weight of the lipid material. In another alternate embodiment, the concentration of alcohol may range from about 1500% to about 2000% by weight of the lipid material.

Table A lists various combinations of lipid material and alcohol that may be used in the processes of the invention.

TABLE A
Substrate Combinations

| Lipid Material | Alcohol |
|---|---|
| vegetable oil | methanol |
| vegetable oil | ethanol |
| vegetable oil | propanol |
| vegetable oil | isopropanol |
| vegetable oil | butanol |
| vegetable oil | isobutanol |
| vegetable oil | alcohol combination |
| soybean oil | methanol |
| soybean oil | ethanol |
| soybean oil | propanol |
| soybean oil | isopropanol |
| soybean oil | butanol |
| soybean oil | isobutanol |
| soybean oil | alcohol combination |
| rapeseed (canola) oil | methanol |
| rapeseed (canola) oil | ethanol |
| rapeseed (canola) oil | propanol |
| rapeseed (canola) oil | isopropanol |
| rapeseed (canola) oil | butanol |
| rapeseed (canola) oil | isobutanol |
| rapeseed (canola) oil | alcohol combination |
| animal fat | methanol |
| animal fat | ethanol |
| animal fat | propanol |
| animal fat | isopropanol |
| animal fat | butanol |
| animal tat | isobutanol |
| animal fat | alcohol combination |
| tallow | methanol |
| tallow | ethanol |
| tallow | propanol |
| tallow | isopropanol |
| tallow | butanol |
| tallow | isobutanol |
| tallow | alcohol combination |
| lard | methanol |
| lard | ethanol |
| lard | propanol |
| lard | isopropanol |
| lard | butanol |
| lard | isobutanol |
| lard | alcohol combination |
| chicken fat | methanol |
| chicken fat | ethanol |
| chicken fat | propanol |
| chicken fat | isopropanol |
| chicken fat | butanol |
| chicken fat | isobutanol |
| chicken fat | alcohol combination |
| fish oil | methanol |
| fish oil | ethanol |
| fish oil | propanol |
| fish oil | isopropanol |
| fish oil | butanol |
| fish oil | isobutanol |
| fish oil | alcohol combination |
| vegetable oil and animal fat/oil | methanol |
| vegetable oil and animal fat/oil | ethanol |
| vegetable oil and animal fat/oil | propanol |
| vegetable oil and animal fat/oil | isopropanol |
| vegetable oil and animal fat/oil | butanol |
| vegetable oil and animal fat/oil | isobutanol |
| vegetable oil and animal fat/oil | alcohol combination |
| algae oil | methanol |
| algae oil | ethanol |
| algae oil | propanol |
| algae oil | isopropanol |
| algae oil | butanol |
| algae oil | isobutanol |
| algae oil | alcohol combination |
| waste vegetable oil (WVU) | methanol |
| waste vegetable oil (WVU) | ethanol |
| waste vegetable oil (WVU) | propanol |
| waste vegetable oil (WVU) | isopropanol |
| waste vegetable oil (WVU) | butanol |
| waste vegetable oil (WVU) | isobutanol |
| waste vegetable oil (WVU) | alcohol combination |
| yellow grease | methanol |
| yellow grease | ethanol |
| yellow grease | propanol |
| yellow grease | isopropanol |
| yellow grease | butanol |
| yellow grease | isobutanol |
| yellow grease | alcohol combination |
| acid oil (soapstock) | methanol |
| acid oil (soapstock) | ethanol |
| acid oil (soapstock) | propanol |
| acid oil (soapstock) | isopropanol |
| acid oil (soapstock) | butanol |
| acid oil (soapstock) | isobutanol |
| acid oil (soapstock) | alcohol combination |

(c) Zirconium(IV) Metal Salt

The esterification process comprises contacting a lipid material with an alcohol in the presence of a zirconium(IV) metal salt conjugated to a solid support. In general, the zirconium(IV) metal salt functions as a catalyst and accelerates the rate of the reaction. The structure of the zirconium(IV) metal salt can and will vary, without departing from the scope of the invention.

In one embodiment of the invention, the zirconium(IV) metal salt may have Formula (I):

ZrX₄ (I)

wherein, X is a halogen atom selected from the group consisting of F, Cl, Br, and I. Non-limiting examples of ZrX₄ compounds include ZrF₄, ZrCl₄, ZrBr₄₁ and ZrI₄. A ZrX₄ compound may also be coordinated with another ligand, such as tetrahydrofuran (THF), N,N-dimethylformamide (DMF), another amide, or water. For example, the zirconium halide may comprise ZrCl₄.(THF)₂, ZrBr₄.(THF)₂, ZrI₄.(THF)₂, ZrCl₄.(DMF)₂, or a similar complex.

In another embodiment, the zirconium(IV) metal salt may have Formula (II):

ZrOX₂.nH₂O (II)

wherein, X is a halogen atom selected from the group consisting of F, Cl, Br, and I; and n is an integer from 1 to about 10. Suitable examples of ZrOX₂.nH₂O compounds include, but are not limited to, ZrOCl₂.6H₂O, ZrOCl₂.8H₂O, and ZrOBr₂.8H₂O. One skilled in the art will appreciate that the number of water molecules coordinated to Zr(IV) may vary. In a preferred embodiment, the number of water molecules may be 8.

In an alternate embodiment, the zirconium(IV) metal salt may have Formula (III):

Zr(XO₄)₄ (III)

wherein, X is a halogen atom selected from the group consisting of F, Cl, Br, and I. A representative example of a Zr(XO₄)₄compound is Zr(ClO₄)₄. A Zr(XO₄)₄compound may and may not be coordinated with THF, DMF, water, or another ligand.

In a further embodiment, the zirconium(IV) metal salt may have Formula (IV):

Zr(OR¹ )₄ (IV)

wherein, R¹ is an acyl group having from 1 carbon atom to about 6 carbon atoms or an alkyl group having from 1 carbon atom to about 6 carbon atoms. Non-limiting examples of Zr(OR)₄ compounds include zirconium(IV) methoxide, zirconium(IV) ethanoxide, zirconium(IV) isopropoxide, zirconium(IV) isobutoxide, zirconium(IV) tetraacetate, and zirconium(IV) tetrapropionate.

In still another embodiment, the zirconium(IV) metal salt may have Formula (V):

Zr(OH)ₘ(OR¹)ₚ (V)

wherein, R¹ is an acyl group having from 1 carbon atom to about 6 carbon atoms or an alkyl group having from 1 carbon atom to about 6 carbon atoms, and m and p are integers from 0 to 4, wherein the sum total of m and p is 4, Examples of Zr(OH)ₘ(OR¹)ₚ compounds include, but are not limited to, zirconium(IV) diacetate dihydroxide and zirconium(IV) triacetatehydroxide.

Without departing from the scope of the invention, the catalyst may comprise more than one the zirconium(IV) metal salt. Furthermore, the zirconium(IV) metal salt may be complexed with another metal salt. Suitable metal salts include iron(III), tin(IV), gallium(III), and hafnium(IV).

In a preferred embodiment, the zirconium(IV) metal salt may be zirconium tetrachloride.

Those skilled in the art will appreciate that the concentration of the zirconium(IV) metal salt catalyst used in the esterification reaction can and will vary, depending upon the source of the lipid material, the temperature of the reaction, and so forth. In one embodiment, the concentration of the catalyst may range from about 0.2% to about 50% by weight of the lipid material. In another embodiment, the concentration of the catalyst may range from about 0.5% to about 35% by weight of the lipid material. In still another embodiment, the concentration of the catalyst may range from about 1% to about 20% by weight of the lipid material.

### (d) Solid Support

In general, a solid support refers to any material that is a solid at ambient temperature, immobilizes the metal salt, and does not react with the lipid material or the alcohol reactants. Suitable solid supports include silicas, alumina, titania, carbondium, zirconia, activated charcoal, zeolites, clays, polymers, ceramics, activated carbon, and porous metal supports. Suitable silicas include silicon dioxide, amorphous silica, and microporous or mesoporous silicas, such as SBA-15, MCM-41, FSM-16. A polymer may be a natural polymer, a synthetic polymer, a semi-synthetic polymer, or a copolymer. Non-limiting examples of polymers include agarose, cellulose, nitrocellulose, methyl cellulose, polyacrylic, polyacrylamide, polyacrylonitrile, polyamide, polyether, polyester, polyethylene, polystyrene, polysulfone, polyvinyl chloride, polyvinylidene, methacrylate copolymer, and polystyrene-vinyl chloride copolymer. The solid support may be a variety of sizes and forms depending upon the embodiment of the invention. For example, the solid support may be beads, microbeads, nanobeads, solid granules, particles, nanoparticles, resins, powders, fibers, nanofibers, nanotubes, gels, sol-gels, areogels, membranes, or a solid surface coated with a solid support. In a preferred embodiment, the solid support may be alumina (i.e., aluminum oxide). Alumina supports of a variety of different sizes and shapes may be used.

Depending upon the particular embodiment, the metal salt may be conjugated to the solid support by a variety of chemical bonds including, but not limited to, covalent bonding, non-covalent bonding, dative bonding, ionic bonding, hydrogen bonding, metallic bonding, or van der Waals bonding. In an exemplary embodiment, the conjugation is via non-covalent bonding. Methods for conjugating a metal salt to a solid support are well known to those skilled in the art. By way of a non-limiting example, the metal salt and the solid support may be mixed in the presence of at least one solvent at room temperature; the mixture may be brought to reflux; or the mixture may be heated to a temperature between about 100° C. to about 300° C. The nature of the solvent can and will vary depending upon the reactants. The duration of the reaction can and will vary, depending upon the temperature and the reactants. Typically, the solvent is removed and the conjugated metal salt/solid support complex is dried before use.

The metal salt may be conjugated directly to the solid support. Alternatively, the metal salt may be conjugated to the solid support by at least one linker. Typically, a linker is a molecule having at least two functional groups, such that the linker is disposed between the solid support and the metal salt. Thus, one functional group of the linker forms an attachment with the solid support. Typically, the linker will be attached to the solid support by a strong covalent bond. And another functional group of the linker forms an attachment with the metal salt by any of the bonding means mentioned above. The composition of the linker, as well as its length, charge, and hydrophobicity, can and will vary depending upon the metal salt, the solid support material, and the intended uses of the supported metal salt.

The weight ratio between the metal salt and the solid support can and will vary depending upon the reactants. The ratio between the metal salt and the solid support may range from about 1:1 to about 1:100.

### (e) Reaction Conditions

The temperature at which the esterification reaction of the invention is conducted may vary. In general, the temperature will be below the flash points of the substrates. In one embodiment, the temperature of the reaction may range from about 25° C. to about 75° C. In another embodiment, the temperature of the reaction may range from about 40° C. to about 70° C. In an alternate embodiment, the temperature of the reaction may be about 45° C. In still another embodiment, the temperature of the reaction may be about 50° C. In still another embodiment, the temperature of the reaction may be about 55° C. In yet another embodiment, the temperature of the reaction may be about 60° C. In a preferred embodiment, the temperature of the reaction may be about 65° C.

The duration of the esterification reaction of the invention can and will vary, depending upon the reaction parameters. Typically, the duration of the reaction will be long enough for the reaction to go to completion, i.e., substantially all of the free fatty acids have been converted into fatty acid esters. Techniques well known in the art, such as gas chromatography (GC), nuclear magnetic resonance (NMR), or mass spectrometry (MS), may be used to determine the completeness of the reaction. In one embodiment, the duration of the reaction may range form about 5 seconds to about 48 hours. In another embodiment, the duration of the reaction may range from about 1 minute to about 24 hours. In still another embodiment, the duration of the reaction may range from about 5 minutes to about 12 hours. In an alternate embodiment, the duration of the reaction may range from about 10 minutes to about 6 hours. In yet another embodiment, the duration of the reaction may range from about 15 minutes to about 4 hours.

The pressure under with the reaction is conducted may vary. The pressure may range from low pressures, such as 40-60 kPa (˜6-9 psia) to high pressures, such as 350-1200 kPa (˜50-175 psia). Typically, however, the reaction may be carried out at atmospheric pressure, which is about 100 kPa (˜14.5 psia).

Typically, the reaction may be performed without an additional organic solvent; i.e., a solvent in addition to the alcohol substrate described above in section (I)(b).

The esterification process of the invention may be conducted in a batch, a semi-continuous, or a continuous mode. The operations may be suitably carried out using a variety of apparatuses and processing techniques well known to those skilled in the art. Furthermore, some of the operations may be omitted or combined with other operations without departing from the scope of the present invention. In a preferred embodiment, the reaction may be performed in a continuous mode of operation. Accordingly, the supported zirconium metal salt may be packed in a catalyst bed for repeated uses in, for example, a continuous stirred tank reactor or in a plug-flow tubular reactor.

### (e) Reaction Products

Upon completion of the esterification reaction, the reaction product solution comprises fatty acid alkyl esters, water, glycerides, and optionally, unreacted alcohol. Depending upon the alcohol or alcohols used in the reaction, the fatty acid alkyl esters may be fatty acid methyl esters, fatty acid ethyl esters, fatty acid propyl esters, fatty acid isopropyl esters, fatty acid butyl esters, fatty acid isobutyl esters, or combinations thereof.

The yield of fatty acid alkyl esters, under optimal reaction conditions, is typically at least about 90%. Depending upon the reaction conditions and other factors, the yield of fatty acid alkyl esters may be at least about 13%, 15%, 20%, 25%, 30%, 35%, 40%, 45%, 50%, 55%, 60%, 65%, 70%, 75%, 80%, 85%, 90%, 95%, 97%, 99%, or 99.5%,

The reaction products may be subjected to at least one additional chemical reaction to convert the glycerides in the solution to fatty acid alkyl esters, as described in section (II). Alternatively, the reaction product solution may be post-treated to remove reaction byproducts and/or impurities. For example, the water in the solution may be removed. Alternatively, the alcohol in the solution may be removed. Distillation and other suitable techniques are well known to those skilled in the art.

The supported zirconium(IV) metal salt catalyst may be recovered, regenerated, and reused. The method used to recover the immobilized catalyst can and will vary, depending mainly upon the mode of operation of the reaction. In a batch reaction, for example, the immobilized catalyst may be recovered by filtration or centrifugation. The catalyst would be retained in a fixed-bed column reactor, however. The supported catalyst may be regenerated for repeated use. For example, the supported zirconium metal salt catalyst may be treated with a zirconium metal salt, washed with a solvent, and optionally, dried. Alternatively, the supported catalyst may be washed with a solvent and, optionally, dried. The number of times the supported catalyst may be reused can and will vary. In general, the yield or efficiency of the reaction decreases with each repeated use. The immobilized catalyst may be used until it is completely spent, i.e., when the percent conversion of free fatty acids into fatty acid esters is less than about 20%, less than about 10%, or less than about 5%. Typically, however, economic considerations will dictate that the immobilized catalyst be replaced prior to becoming completely spent. For a particular system, those skilled in the art will be readily able to determine when repeated use of the supported catalyst is no longer economically viable in view of increased fatty acid ester losses and the capital expenditure necessary for replacement of the catalyst.

### (I) A Combined Esterification and Transesterification Process for Forming a Biodiesel Solution

Another aspect of the present invention provides a process for forming a biodiesel solution. This process combines the esterification process detailed above in section (I) and a transesterification process, which is detailed below. During the esterification reaction, the free fatty acids in the lipid material react with an alcohol in the presence of a zirconium(IV) metal salt catalyst to form fatty acid esters and water. During the transesterification reaction, the glycerides in the lipid material react with the alcohol in the presence of a transesterification catalyst to form fatty acid esters and glycerol. Upon completion of the combined process, therefore, the biodiesel solution comprises fatty acid alkyl esters, glycerol, and water.

Lipid materials and alcohols suitable for use in this process were described above in section (I)(a) and section (I)(b), respectively. Suitable zirconium(IV) metal salt catalysts and reaction conditions for the esterification reaction were detailed above in sections (I)(c)-(e). In general, the esterification reaction solution is contacted with a transesterification catalyst (see below) without any pretreatment. The unreacted alcohol in the esterification reaction solution may be used for the transesterification reaction. Alternatively, the unreacted alcohol in the esterification reaction solution may be removed, and fresh alcohol (i.e., the same or a different one) may be added to the solution.

### (a) N-heterocyclic Carbene Catalyst

The transesterification reaction may be catalyzed by an N-heterocyclic carbene. N-Heterocyclic carbenes suitable for use in the invention generally have formula (VI) or formula (VII):

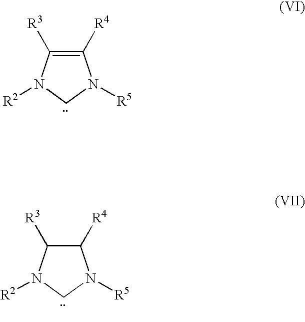

wherein, R² and R⁵ are independently selected from the group consisting of a hydrocarbyl group having from 1 carbon atom to about 12 carbon atoms and a substituted hydrocarbyl group having from 1 carbon atom to about 12 carbon atoms; and R³ and R⁴ are independently selected from the group consisting of a hydrogen atom, a halogen atom, and a hydrocarbyl group having from 1 carbon atom to about 6 carbon atoms.

N-Heterocyclic carbenes suitable for use in the present invention are detailed in Table B.

TABLE B
N-Heterocyclic carbene compounds.

| Compound | | |
|---|---|---|
| Number | Formula Type | Chemical Structure |
| 1 | A compoundhaving Formula (VI) | 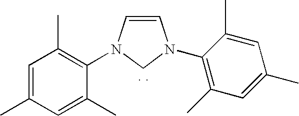 |
| 2 | A compoundhaving Formula (VI) | 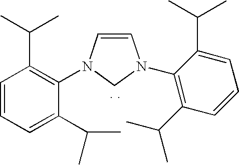 |
| 3 | A compoundhaving Formula (VI) | 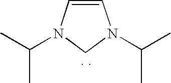 |
| 4 | A compoundhaving Formula (VI) | 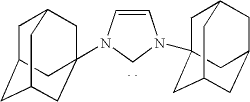 |
| 5 | A compoundhaving Formula (VI) | 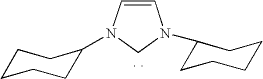 |
| 6 | A compoundhaving Formula (VI) | 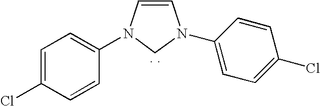 |
| 7 | A compoundhaving Formula (VI) | 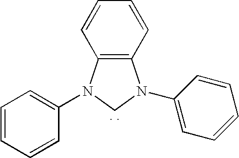 |
| 8 | A compoundhaving Formula (VI) | 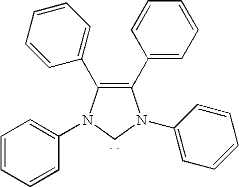 |
| 9 | A compoundhaving Formula (VI) | 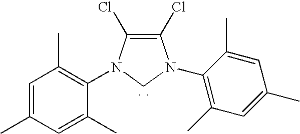 |
| 10 | A compoundhaving Formula (VI) | 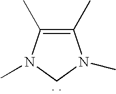 |
| 11 | A compoundhaving Formula (VI) | 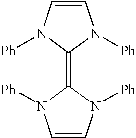 |
| 12 | A compoundhaving Formula (VI) | 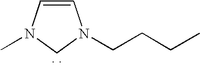 |
| 13 | A compoundhaving Formula (VII) | 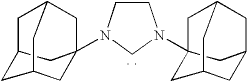 |
| 14 | A compoundhaving Formula (VII) | 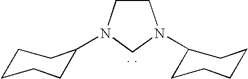 |
| 15 | A compoundhaving Formula (VII) | 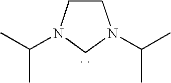 |
| 16 | A compoundhaving Formula (VII) | 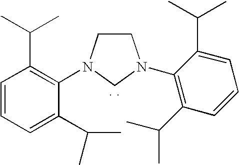 |
| 17 | A compoundhaving the Formula (VII) | 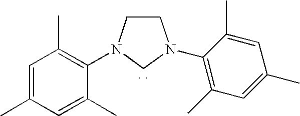 |

In one embodiment, the N-heterocyclic carbene catalyst may be 1,3-bis(2,4,6-trimethylphenyl)imidazol-2-ylidene (IMes, Compound 1 in Table B). In another embodiment, the N-heterocyclic carbene catalyst may be 1,3-bis(2,6-diisopropylphenyl)imidazol-2-ylidene (IprP, Compound 2 in Table B). In still another embodiment, the N-heterocyclic carbene catalyst may be 1,3-diisopropylimidazol-2-ylidene (Ipro, Compound 3 in Table B). In a further embodiment, the N-heterocyclic carbene catalyst may be 1,3-bis(1-adamantyl)imidazol-2-ylidene (IAda, Compound 4 in Table B). In still another embodiment, the N-heterocyclic carbene catalyst may be 1,3-dicyclohexylimidazol-2-ylidene (Icy, Compound 5 in Table B). In another embodiment, the N-heterocyclic carbene catalyst may be 1-butyl-3-methylimidzol-2-ylidene (IBuM, Compound 12 in Table B). In still a further embodiment, the N-heterocyclic carbene catalyst may be 1,3-diisopropylimidzolin-2-ylidene (SPro, Compound 15 in Table B). Without limiting the scope of the invention, the N-heterocyclic carbene catalyst may be a metal derivative, a dimer, or a combination of any of the aforementioned compounds.

The concentration of N-heterocyclic carbene catalyst used in the transesterification reaction can and will vary. In one embodiment, the concentration of the N-heterocyclic carbene catalyst may range from about 0.02% to about 40% by weight of the starting lipid material. In another embodiment, the concentration of the N-heterocyclic carbene catalyst may range from about 0.5% to about 30% by weight of the starting lipid material. In still another embodiment, the concentration of the N-heterocyclic carbene catalyst may range from about 1% to about 20% by weight of the starting lipid material.

The N-heterocyclic carbene may be conjugated to a solid support, either directly or via a linker, as detailed above in section (I)(d). In one embodiment, an N-heterocyclic carbene is covalently bound to a divinylbenzene-crosslinked polystyrene-vinyl chloride co-polymer. In another embodiment, an N-heterocyclic carbene is covalently bond to a silica support. In yet another embodiment, an N-heterocyclic carbene is conjugated to a silica support via a linear alkly linker comprising from about 4 carbon atoms to about 8 carbon atoms.

### (b) Alkaline Catalyst

The transesterification reaction may also be catalyzed by an alkaline catalyst. In one embodiment, the alkaline catalyst may be sodium hydroxide. In another embodiment, the alkaline catalyst may be potassium hydroxide. In still another embodiment, the alkaline catalyst may be sodium methoxide. In an alternate embodiment, the alkaline catalyst may be potassium methoxide.

The concentration of the alkaline catalyst can and will vary, depending upon the starting lipid material and other factors. In general, the concentration of the alkaline catalyst may range from about 1% to about 25% by weight of the starting lipid material.

### (c) Reaction Conditions

The temperature at which the transesterification reaction is conducted can and will vary, depending upon the substrates and the catalyst utilized. In one embodiment, the temperature of the reaction may range from about 15° C. to about 75° C. In an alternate embodiment, the temperature of the reaction may range from about 18° C. to about 40° C. In another embodiment, the temperature of the reaction may range from about 20° C. to about 30° C. In a preferred embodiment, the temperature of the reaction may be at ambient temperature (i.e., about 21°-23° C.).

The duration of the transesterification reaction can and will vary, depending upon a variety of factors. Typically, the duration of the reaction will be long enough for the reaction to go to completion, i.e., all of the glycerides have been converted into fatty acid alkyl esters. Techniques well known in the art, such as gas chromatography (GC), nuclear magnetic resonance (NMR), or mass spectrometry (MS), may be used to determine the completeness of the reaction. In one embodiment, the duration of the reaction may range from about 5 seconds to about 48 hours. In another embodiment, the duration of the reaction may range from about 30 seconds to about 24 hours. In still another embodiment, the duration of the reaction may range from about 1 minute to about 3 hours. In an alternate embodiment, the duration of the reaction may range from about 5 minutes to about 2 hours.

The pressure under with the reaction is conducted may vary. The pressure may range from low pressures, such as 40-60 kPa (˜6-9 psia) to high pressures, such as 350-1200 kPa (-50-175 psia). Typically, however, the reaction may be carried out at atmospheric pressure, which is about 100 kPa (-14.5 psia).

The reaction will generally be carried out without an additional organic solvent; that is, a solvent in addition to the alcohol substrate.

The transesterification process may be conducted in a batch, a semi-continuous, or a continuous mode, as detailed above.

### (d) Reaction Products

Upon completion of the combined esterification/transesteritication process, a biodiesel solution is formed. The biodiesel solution comprises fatty acid alkyl esters, glycerol, water, and optionally, unreacted alcohol. Depending upon the alcohol or alcohols used in the reactions, the fatty acid alkyl esters may be fatty acid methyl esters, fatty acid ethyl esters, fatty acid propyl esters, fatty acid isopropyl esters, fatty acid butyl esters, fatty acid isobutyl esters, or combinations thereof.

The yield of fatty acid alkyl esters, under optimal reaction conditions, is typically at least about 90%. Depending upon the reaction conditions and other factors, the yield of fatty acid alkyl esters may be at least about 13%, 15%, 20%, 25%, 30%, 35%, 40%, 45%, 50%, 55%, 60%, 65%, 70%, 75%, 80%, 85%, 90%, 95%, 97%, 99%, or 99.5%.

The biodiesel solution may be treated to remove reaction byproducts and/or impurities. In one embodiment, the post treatment process may include distillation to remove the alcohol from the biodiesel solution. In another embodiment, the post treatment process may include vacuum drying to remove water from the biodiesel solution. In yet another embodiment, the post treatment process may also include degumming to remove phosphatides or other solid residues from the biodiesel solution. In still another embodiment, the biodiesel solution may be washed with warm water to remove residual catalyst. In yet an alternate embodiment, the post treatment process may include the purification and fraction of fatty acid esters via distillation or vacuum distillation processes. One skilled in the art will know which process(es) to perform and how to perform them.

The fatty acid alkyl esters formed by the processes of the present invention may be commercially useful for fuel compositions, lubricants, emulsifiers, plasticizers, intermediates for the production of products, such as soaps, detergents, or fragrances, and so forth. In an exemplary embodiment, the fatty acid ester may be used as a fuel composition. The fuel composition may be a biodiesel. Alternatively, the fuel composition may be a blend of a petroleum based diesel fuel and biodiesel.

### Definitions

To facilitate understanding of the invention, a number of terms and abbreviations, as used herein, are defined below:

The term “acyl” denotes a radical having the general formula RCO—, provided after the removal of a hydroxyl group from an organic acid. Examples of acyl radicals include alkanoyl and aroyl radicals. Examples of lower alkanoyl radicals include formyl, acetyl, propionyl, butyryl, isobutyryl, valeryl, isovaleryl, pivaloyl, hexanoyl, and trifluoroacetyl.

The term “alkyl” embraces linear, cyclic, or branched hydrocarbon radicals having one carbon atom to about twenty carbon atoms. More preferred alkyl radicals are “lower alkyl” radicals having one carbon atom to about six carbon atoms. Examples of such radicals include methyl, ethyl, n-propyl, isopropyl, n-butyl, isobutyl, sec-butyl, tert-butyl, pentyl, iso-amyl, hexyl, and the like.

The term “biodiesel,” as used herein, refers to a composition of alkyl monoesters of fatty acids derived from a biological material.

As used herein, “esterification” refers to a chemical process of condensing fatty acids with an alcohol in the presence of a catalyst.

The term “fatty acid,” as used herein, refers to any of a large group of organic acids made up of molecules containing a carboxyl group (—COOH) at the end of a usually unbranched hydrocarbon chain. The hydrocarbon chain may have from about 4 to about 24 carbon atoms, or more specifically, from about 12 to about 22 carbons. The hydrocarbon chain may be saturated or unsaturated.

The term “free fatty acid,” as used herein, refers to the fatty acid product upon breakage of an ester link of a glyceride.

The term “glyceride,” as used herein, refers to esters formed from glycerol and fatty acids. Glycerol has three hydroxyl functional groups that may be esterified with one, two, or three fatty acids to form a monoglyceride, a diglyceride, or a triglyceride.

As used herein, the terms “hydrocarbon” and “hydrocarbyl” describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties that are substituted with at least one atom other than carbon, including moieties in which a carbon chain atom is substituted with a heteroatom such as nitrogen, oxygen, silicon, phosphorous, boron, sulfur, or a halogen (i.e., fluorine, chlorine, bromine, iodine) atom. These substituents also include carbocycle, aryl, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters, and ethers.

The term “transesterification,” as used herein, refers to the chemical process of exchanging the alkoxy group of an ester compound by another alcohol in the presence of a catalyst.

### EXAMPLES

The following examples are given by way of illustration only and therefore should be constructed to limit the scope of the present invention.

Soy oil was purchased from a local co-op or supermarket stores and was used without further purification. Methanol and all other chemicals were obtained from Acros Organics (Somerville, N.J.) or Aldrich (Milwaukee, Wis.) and used as received without further purification. Pluronic P-123 was a gift from BASF Corporation (Florham Park, N.J.). Alumina matrices were provided by Saint-Gobain NorPro (Stow, Ohio). ¹H NMR analyses were performed on a Varian VXR-300 system (Palo Alto, Calif.) with an Oxford wide-bore magnet, and the chemical shifts were reported in parts per million (ppm) downfield relative to tetramethylsilane using the residual proton resonance of solvents as the references (¹H NMR): CDCl₃ δ 7.27; CD₂Cl₂ δ 5.32 and (¹³C NMR): CDCl₃ δ 77.2; CD₂Cl₂ δ 54.0.

### Example 1

### Synthesis of Mesoporous Silica (SBA-1 5).

SBA-15, an ordered porous silicate, was synthesized using an adaptation of the method of Zhao et al. (J. Am. Chem. Soc. 1998, 120, 6024-6036). Pluronic P-123 (4 g) was added to 2 M HCl (125 mL) at ambient temperature (25° C.). The temperature of the mixture was raised to 50° C. After 2 h, the solution was cooled to 40-45° C., and then 8.54 g of tetraethyl orthosilicate (TEOS) was added. Precipitation was observed. The solution was stirred for 22 h at 40-45° C. Then, the temperature was raised to 95° C. After 24 h, the solution was cooled to ambient temperature, and the precipitate was collected and dissolved in 200 mL of ethanol. After refluxing for 12 h, the ethanol solution mixture was cooled to room temperature. The precipitate was filtered and suspended in another 200 mL of fresh ethanol. The mixture was brought to reflux. After 12 h, the mixture was cooled to room temperature. The precipitate was filtered and dried under vacuum for 12 h. Examination of the resultant SBA-15 matrix by transmission electron microscopy (TEM) revealed an ordered porous structure, with pore diameters of about 10 nm.

### Example 2

### Synthesis of Immobilized Metal Salt Complexes

Synthesis of ZrOCl₂.8H₂O and SBA-15 complex: SBA-15 (112 mg) and ZrOCl₂.8H₂O (129 mg) were mixed in 15 mL of toluene and brought to reflux. After 2 h, the mixture was cooled down to ambient temperature. The solvent was evaporated in vacuo and the residue was washed with toluene (50 mL x 3) and methanol (50 mL×3) and air-dried to give a powder (210 mg). Elemental analysis confirmed the presence of Zr in the silica.

Synthesis of ZrCl₄ and SBA-15 complex: a mixture of ZrCl₄ (92 mg) and SBA-15 (112 mg) in 15 mL of methanol was brought to reflux. After 30 min, the solvent was evaporated and the residue was washed with toluene (50 mL×3) and air-dried. Elemental analyses were employed to confirm the structure.

Synthesis of ZrO₂ and SBA-15 complex: similar to the procedure of ZrCl₄ above: ZrO₂(49 mg) and SBA-15 (112 mg).

Synthesis of Zr(SO₄)₂ and SBA-15 complex: similar to the procedure of ZrCl₄ above: Zr(SO₄)₂ (113 mg) and SBA-15 (112 mg).

Synthesis of ZrSiO₄ and SBA-15 complex; similar to the procedure of ZrCl₄ above: ZrSiO₄ (73 mg) and SBA-15 (112 mg).

Synthesis of Zr(OC₄H₉)₄ and SBA-15 complex: similar to the procedure of ZrCl₄ above: Zr(OC₄H₉)₄ (152 mg) and SBA-15 (112 mg).

Synthesis of SnCl₄.5H₂O and SBA-15 complex: similar to the procedure of ZrCl₄ above: SnCl₄.5H₂O (140 mg) and SBA-15 (112 mg).

Synthesis of TiCl₄ and SBA-15 complex: similar to the procedure of ZrCl₄ above: TiCl₄ (75 mg) and SBA-15 (112 mg).

Synthesis of FeCl₃.6H₂O and SBA-15 complex: similar to the procedure of ZrCl₄ above: FeCl₃.6H₂O (108 mg) and SBA-15 (112 mg).

Synthesis of ZrCl₄, SnCl₄.5H₂O and SBA-15 complex: a mixture of ZrCl₄ (92 mg), SnCl₄.5H₂O (40 mg), and SBA-15 (112 mg) in 10 mL of methanol was brought to reflux. After 30 min, the solvent was evaporated and the residue was washed with toluene (50 mL×3) and air-dried. Elemental analyses were employed to confirm the structure.

Synthesis of ZrCl₄ and titania complex: a mixture of ZrCl₄ (230 mg) and titania (400 mg) in 10 mL of toluene was brought to reflux. After 4 h, the mixture was cooled down to room temperature. Solvent was evaporated in vacuo and the residue was washed with toluene (50 mL×3) and methanol (50 mL×3) sequentially. Elemental analysis experiments were adopted to confirm the structure of the composite.

Synthesis of ZrCl₄ and carbondium complex: similar to the aforementioned synthesis of the composite of ZrCl₄ and titania: ZrCl₄ (230 mg) and carbondium (200 mg).

Synthesis of ZrCl₄ and activated carbon complex: similar to the aforementioned synthesis of the composite of ZrCl₄ and titania: ZrCl₄ (230 mg) and activated carbon (60 mg).

Synthesis of ZrCl₄ and alumina complexes: a mixture of ZrCl₄ (400 mg) and an alumina support (187 mg) (e.g., SA6176 from Saint-Gobain NorPro) in 10 mL of CHCl₃ was brought to reflux. After 4 h, the mixture was cooled down to ambient temperature. The composite was filtered and washed with CHCl₃ (50 mL×3) and methanol (50 mL×3) sequentially, and air-dried. About 218 mg of the composite was recovered. Alumina supports of other shapes and sizes were loaded similarly with ZrCl₄.

### Example 3

### Catalytic Activity of Metal salt/SBA-15 Complexes

The ability of the different metal salt/SBA-15 complexes synthesized in Example 2 to catalyze the esterification of a free fatty acid (FFA) was tested in batch reactions. For this, a mixture of oleic acid (0.282 g) and methanol (4 g) was mixed with each of the metal salt/SBA-15 complexes (28 mg). Each mixture was brought to reflux (65° C.). At fixed time intervals, a small amount of the mixture was removed from each reactor. After removal of the solvent, the amount of the product (i.e., oleate methyl ester) was estimated by ¹H NMR (see Table 1). The most efficient catalysts were ZrOCl₂.8H₂O, ZrCl₄, Zr(OC₄H₉)₄, and a combination of ZrCl₄+SnCl₄; each produced nearly 100% yield after a 45 min reaction.

TABLE 1
Esterification of oleic acid.

| | |
|---|---|
| Metal salt | Yield (%) |

| conjugated to SBA-15 | 15 min | 45 min |
|---|---|---|
| ZrOCl₂•8H₂O | 88 | >99 |
| ZrCl₄ | 85 | >99 |
| ZrO₂ | trace | trace |
| Zr(SO₄)₂ | trace | trace |
| ZrSiO₄ | trace | trace |
| Zr(OC₄H₉)₄ | 85 | >99 |
| SnCl₄•5H₂O | 45 | 65 |
| TiCl₄ | 63 | 77 |
| FeCl₃•6H₂O | 9 | 15 |
| ZrCl₄ + SnCl₄ | 89 | >99 |

### Example 4

### Effectiveness of Metal salt/SBA-15 Complexes to Catalyze the Esterification of a Free Fatty Acid in Soy Oil

The ability of some of the metal salts/SBA-15 complexes to catalyze the esterification of a free fatty acid (FFA) in soy oil was also tested. For this, 17.92 g of commercial RBD (refined, bleached, and deodorized) soy oil was mixed with 5.65 g of oleic acid. About 1.78 g of this mixture was added to a mixture of methanol (33 my) and a metal salt/SBA-15 complex (28 mg). Each mixture was brought to 65° C. At fixed time intervals, a small amount of the mixture was removed from each reaction. The solvent was removed and the amount of FFA remaining was estimated via titration experiments using the AOCS method Ca 5a-40 (Official Methods and Recommended Practices of the AOCS). The final product was analyzed by ¹H NMR, and the yields are presented in Table 2. The zirconium metal salt complexes were the best catalysts.

TABLE 2
Esterification of oleic acid in soy oil.

| | |
|---|---|
| Metal salt | Yield (%) |

| conjugated to SBA-15 | 30 min | 60 min |
|---|---|---|
| ZrOCl₂•8H₂O | 84 | >99 |
| ZrCl₄ | 86 | >99 |
| Zr(OC₄H₉)₄ | 81 | >99 |
| SnCl₄•5H₂O | 23 | 57 |
| ZrCl₄ + SnCl₄ | 92 | >99 |

### Example 5

### Catalytic Activity of ZrCl₄ Immobilized Complexes

The ability of ZrCl₄ complexed to different solid matrices to catalyze the esterification of FFA in soy oil was determined. Each of the different ZrCl₄ complexes was added to a mixture of methanol (5 mL), RBD soy oil (0.896 g), and oleic acid (0.282 g). The reaction was carried out at ambient temperature (25° C.). After 2 h, the solvent was removed in vacuo and the amount of FFA remaining was estimated via titration experiments (as described in Example 4). The final product was analyzed by ¹H NMR, and the yields are presented in Table 3. Each of the solid-supported ZrCl₄ complexes was a good catalyst.

TABLE 3
Esterification of oleic acid in soy oil in the presence of supported
ZrCl₄ complexes.

| Supporting Matrix | Titania | Carbondium | Activated Carbon |
|---|---|---|---|
| Composite amount (mg) | 59 | 46 | 30 |
| Yield (%) | 77 | 72 | 88 |

### Example 6

### Synthesis of ZrCl₄/hydrophobic phenyl-modified SBA-15 Complex

To increase the efficiency of the supported catalyst, SBA-15 was modified to increase its hydrophobicity. To make a phenyl-modified supported metal catalyst, SBA-15 (7 9) and ZrCl₄ (5.75 g) were suspended in 100 mL of toluene. The solution was brought to reflux. After 2 h, the mixture was cooled to room temperature (25° C.). Phenyltrimethoxysilane (3.5 g) was added to the solution and the mixture was brought to reflux. After 2 h, the solvent was removed in vacuo and the residue was repeatedly washed with toluene (50 mL×3) and air-dried to give rise to powders (15.4 g). Elemental analysis using atomic absorption confirmed the presence of Zr and Si in the powders.

### Example 7

### Effect of Temperature on the Esterification of FFA in Soy Oil

Esterification reactions were performed at different temperatures in the presence of the ZrCl₄/phenyl SBA-15 complex. For this, 28 mg of the ZrCl₄/phenyl SBA-15 complex from Example 6 was added to a mixture of oleic acid (0.282 g), soy oil (0.1 g), and 5 mL of methanol. Reactions were conducted at room temperature (25° C.), 40° C., and 65° C. At regular time intervals, a small amount of the sample was removed from each reaction system and subjected to ¹H NMR analysis and titration experiments as described above (after the removal of the catalyst and solvent). Table 4 presents the results. The reactions went to completion in about 12 h at 25° C., about 3.5 h at 40° C., and about 50 min at 65° C.

TABLE 4
Esterification of oleic acid in soy oil at different temperatures.

| 25° C. | | 40° C. | | 65° C. | |
|---|---|---|---|---|---|
| Reaction | | Reaction | | Reaction | |
| time (h) | Yield (%) | time (h) | Yield (%) | time (min) | Yield (%) |
| 0.5 | 14 | 0.5 | 34 | 5 | 22 |
| 1.0 | 25 | 1.37 | 68 | 10 | 44 |
| 1.5 | 34 | 2.0 | 84 | 15 | 58 |
| 2.5 | 51 | 2.67 | 93 | 25 | 79 |
| 3.5 | 60 | 3.5 | >99 | 35 | 90 |
| 4.83 | 76 | | | 50 | >99 |
| 8.5 | 93 | | | | |
| 12 | >99 | | | | |

### Example 8

### Comparison of the Catalytic Activities of ZrCl₄/SBA-15 Complex and ZrCl₄/phenyl SBA-15 Complex

The effectiveness of the ZrCl/SBA-15 and ZrCl₄/phenyl SBA-15 complexes to catalyze the esterification of FFA in soy oil were compared. Parallel reactions were set up; 1) 28 mg of the ZrCl₄/SBA-15 complex (from Example 2) was added to a mixture of oleic acid (0.282 g), soy oil (0.1 g), and 5 mL of methanol; and 2) 44 mg of the ZrCl₄/phenyl SBA-15 complex was added to a similar mixture of substrates. Both reactions had the same amount of Zr (IV) salt based on theoretical calculations. The mixtures were brought to reflux (65° C.). At a regular time intervals, a small amount of the sample was removed from the reaction system and subjected to ¹H NMR and titration analyses. The results are presented in Table 5. The ZrCl₄/phenyl SBA-15 complex was a more efficient catalyst.

TABLE 5
Esterification of oleic acid in soy oil.

| | |
|---|---|
| Reaction | Yield (%) |

| time (min) | ZrCl₄/SBA-15 | ZrCl₄/phenyl SBA-15 |
|---|---|---|
| 5 | 15 | 22 |
| 10 | 31 | 44 |
| 15 | 44 | 58 |
| 25 | 64 | 79 |
| 35 | 80 | 90 |
| 50 | 91 | >99 |

### Example 9

### Esterification of FFA in Soy Oil using a Fixed-Bed Column Reactor Packed with ZrCl₄phenyl SBA-15 Complex

A glass chromatography column with an internal diameter of 3.8 cm was packed with the ZrCl₄/phenyl-modified SBA-15 complex. The catalyst bed was about 5.5 cm in height. The bottom of the column and the top of the catalyst bed were also packed with uncomplexed silica gels (60 Å, 40-63 μm diameter); the height of the bottom and top uncomplexed beds were about 6.3 cm and 3.8 cm, respectively. A mixture of oleic acid (7 g), soy oil (0.5 g), and methanol (50 mL) was pumped through the column at a flow rate of about 0.34-0.37 mL/min. The contact time of the reaction mixture with the catalyst bed was around 35 min. Column fractions were collected and analyzed by ¹H NMR and titration experiments, as described above. It was found that over 99% of the oleic acid was esterified to its methyl ester.

### Example 10

### Comparison of the Catalytic Activities of ZrCl₄/Alumina Complex and Unsupported ZrCl₄.

Parallel reactions with either a supported or an unsupported catalyst were carried out in batch reactors. The catalysts were recovered after the reactions and reused. For the supported catalyst reaction, 0.28 g of the ZrCl₄/alumina (Al₂O₃) complex was added to a mixture oleic acid (2.82 g), soy oil (0.5 g), and methanol (1.6 g). It was estimated that the complex contained about 23 mg of ZrCl₄. The reaction was brought to reflux. After 1 h, the mixture was cooled down to ambient temperature. The solvent was evaporated in vacua and the catalyst complex was recovered by filtration. The solvent was evaporated in vacua, and ¹H NMR and titration experiments, as described above, were used to estimate the reaction yield. The recovered catalyst was washed with methanol (50 mL×3) and reused (up to three times).

For the unsupported catalyst, 23 mg of ZrCl₄ was added to a mixture of oleic acid (2.82 g), soy oil (0.5 g), and methanol (1.6 g). The reaction was brought to reflux. After 1 h, the mixture was cooled down to ambient temperature. The solvent was evaporated in vacua. Filtration partially recovered the catalyst. The solvent was evaporated in vacua, and the reaction yield was estimated as described above. The recovered catalyst was reused in another reaction. To minimize the loss of the recovered catalyst, the filter paper was cut into small pieces and added to the next reaction (along with the recovered catalyst).

The results are presented in Table 6. Although the yields of the reactions catalyzed by the supported catalyst were lower, the supported catalyst could be recycled and reused repeatedly with no loss in activity. In contrast, the unsupported catalyst lost all activity after about three uses.

TABLE 6
Catalytic activity of recycled supported
and unsupported catalyst.

Yield (%)

| | ZrCl₄/alumina | |
|---|---|---|
| Reaction | complex | ZrCl₄ |
| 1^{st} | 17 | 76 |
| 2^{nd} | 19 | 64 |
| 3^{rd} | 17 | 38 |
| 4^{th} | 18 | Not detectable |

### Example 11

### Esterification of FFA in Soy Oil Using a Fixed-Bed Column Reactor Packed with ZrCl₄/Alumina Complex

A glass chromatography column with an internal diameter of 1.6 cm was packed with the ZrCl₄/alumina complex (1.6 mm diameter pellets). The height of the catalyst bed was 24 cm, and the amount of ZrCl₄ was estimated to be 1 g in 12.2 g of alumina pellets. The catalyst bed was warmed to 55° C. A mixture of oleic acid (20 g), soy oil (1 g), and methanol (11.3 g) was pumped through the catalyst bed at a flow rate of 3.5 mL/min. The contact time of the reaction mixture with the catalyst bed was 1.0 min. The reaction yields and reaction products were analyzed by titration experiments and ¹H NMR analyses, as described above. The conversion of oleic acid into oleate methyl ester was estimated to be 13%. A repeat experiment performed under similar conditions also led to a yield of 13.3%. Optimization of the reaction conditions or optimization of the synthesis of the ZrCl₄/alumina complex should improve the yield, however.

### Example 12

### Coupled Esterification and Transesterification Reactions for Converting both FFA and Triglycerides into Fatty Acid Methyl Esters

First, an esterification reaction was performed by adding 1.68 g of the ZrCl₄/alumina complex to a mixture of oleic acid (2.82 g), soy oil (0.5 g), and methanol (1.6 g). The batch reaction was brought to reflux. After 2 h, the mixture was cooled down to ambient temperature. The solvent was evaporated in vacuo and the supported catalyst complex was removed by filtration. It was estimated with ¹H NMR and titration experiments that over 99% of FEA was converted to fatty acid methyl esters.

Second, a transesterification reaction was performed with the esterification reaction mixture. Two different transesterification catalysts were used (an alkaline catalyst and a N-heterocyclic carbene catalyst). To catalyze the reaction with a alkaline catalyst, the esterification reaction mixture was treated with NaOMe (10 mg). The mixture was stirred at ambient temperature for 1 h, and then the reaction mixture was separated into two phases. Excessive methanol was removed in vacuo and the two layers were separated. ¹H NMR experiments and the glycerine analysis (AOCS method Ca14-56) confirmed that over 89% of triglycerides were converted into fatty acid methyl esters.

Alternatively, the esterification reaction mixture was treated with the N-heterocyclic carbene, 1-butyl-3-methylimidzol-2-ylidene. This catalyst was synthesized by dissolving about 0.8 eq. of potassium tert-butoxide (8.9 mg) and 1 eq. of 1-butyl-3-methylimidazolium salt in 2 mL of dry tetrahydrofuran (THF), After 1 h under argon protection, the solvent was removed and the resulting 1-butyl-3-methylimidzol-2-ylidene was added to the esterification reaction mixture. After 1 h, the reaction mixture was separated into two phases. Excessive methanol was removed in vacuo and the two layers were separated. ¹H NMR and glycerine analyses confirmed that over 92% of the triglycerides were converted into fatty acid methyl esters.

What is claimed is:

1. A process for forming a fatty acid alkyl ester, the process comprising contacting a lipid material with an alcohol in the presence of a zirconium(IV) metal salt conjugated to a solid support and at a temperature from about 25° C. to about 75° C., whereby the alcohol reacts with a free fatty acid in the lipid material to form the fatty acid alkyl ester.

2. The process of claim 1, wherein the lipid material comprises free fatty acids and glycerides.

3. The process of claim 2, wherein the lipid material is selected from the group consisting of a vegetable oil, an animal fat, an algae oil, a food-based lipid waste, an industrial lipid waste, and a combination thereof.

4. The process of claim 3, wherein the lipid material is pretreated with a process selected from the group consisting of degumming, vacuum drying, steam distilling, bleaching, and a combination thereof.

5. The process of claim 1, wherein the alcohol is a short chain alcohol having from one carbon atom to about four carbon atoms.

6. The process of claim 5, wherein the short chain alcohol is selected from the group consisting of methanol, ethanol, propanol, isopropanol, butanol, isobutanol, and a combination thereof.

7. The process of claim 1, wherein the concentration of the alcohol is from about 1% to about 2000% by weight of the lipid material.

8. The process of claim 1, wherein the zirconium(IV) metal salt is a compound having a formula selected from the group consisting of ZrX₄, ZrOX₂.nH₂O, Zr(XO₄)₄, Zr(OR¹)₄, and Zr(OH)ₘ(OR¹)ₚ,
wherein:
X is a halogen atom selected from the group consisting of F, Cl, Br, and I;
n is an integer from 1 to about 10;
R¹ is an acyl group having from one carbon atom to about six carbon atoms or an alkyl group having from one carbon atom to about six carbon atoms; and
m and p are integers from 0 to 4, wherein the sum total of m and p is 4.

9. The process of claim 8, wherein the ZrX₄ or Zr(XO₄)₄ compound is coordinated with another ligand selected from the group consisting of tetrahydrofuran, N,N-dimethylformamide, and water.

10. The process of claim 8, wherein the zirconium(IV) metal salt is complexed with another metal salt selected from the group consisting of iron(III), tin(IV), gallium(III), and hafnium(IV).

11. The process of claim 1, wherein the solid support is selected from the group consisting of silicas, alumina, titania, carbondium, zirconia, activated charcoal, zeolites, clays, polymers, ceramics, activated carbon, porous metal supports, and a combination thereof.

12. The process of claim 1, wherein the zirconium(IV) metal salt is conjugated to the solid support by non-covalent bonding.

13. The process of claim 1, wherein the concentration of the zirconium(IV) metal salt is from about 0.2% to about 50% by weight of the lipid material.

14. The process of claim 1, wherein the concentration of the zirconium(IV) metal salt is from about 1% to about 20% by weight of the lipid material.

15. The process of claim 1, wherein the temperature is from about 50° C. to about 70° C.

16. The process of claim 1, wherein the temperature is about 65° C.

17. The process of claim 1, wherein the process is conducted in a mode selected from the group consisting of batch, semi-continuous, and continuous.

18. The process of claim 1, wherein the lipid material is soybean oil, the alcohol is methanol, the zirconium(IV) metal salt is zirconium tetrachloride, the solid support is alumina, and the temperature is 65° C.

19. The process of claim 1, wherein the conversion of free fatty acids to fatty acid alkyl esters is from about 13% to over 99%.

20. The process of claim 1, wherein the conversion of free fatty acids to fatty acid alkyl esters is at least about 90%.

21. A process for forming a biodiesel solution, the process comprising:
(a) contacting a lipid material with an alcohol in the presence of a first catalyst to form a first solution, wherein the lipid material comprises free fatty acids and glycerides, and the first catalyst comprises a zirconium(IV) metal salt; and
(b) contacting the first solution with a second catalyst selected from the group consisting of an N-heterocyclic carbene and an alkaline agent to form the biodiesel solution.

22. The process of claim 21, wherein step (a) is performed at a temperature from about 25° C. to about 65° C. and step (b) is performed at a temperature from about 25° C. to about 40° C.

23. The process of claim 21, wherein the lipid material is selected from the group consisting of a vegetable oil, an animal fat, an algae oil, a food-based lipid waste, an industrial lipid waste, and a combination thereof.

24. The process of claim 23, wherein the lipid material is pretreated with a method selected from the group consisting of degumming, vacuum drying, steam distilling, bleaching, and a combination thereof.

25. The process of claim 21, wherein the alcohol is a short chain alcohol having from one carbon atom to about four carbon atoms.

26. The process of claim 25, wherein the short chain alcohol is selected from the group consisting of methanol, ethanol, propanol, isopropanol, butanol, and isobutanol.

27. The process of claim 21, wherein the concentration of the alcohol is from about 1% to about 2000% by weight of the lipid material.

28. The process of claim 21, wherein the zirconium(IV) metal salt is a compound having a formula selected from the group consisting of ZrX₄, ZrOX₂.nH₂O, Zr(XO₄)₄, Zr(OR¹)₄, and Zr(OH)ₘ(OR¹)ₚ,
wherein:
X is a halogen atom selected from the group consisting of F, Cl, Br, and I;
n is an integer from 1 to 10;
R¹ is an acyl group having from one carbon atom to about six carbon atoms or an alkyl group having from one carbon atom to about six carbon atoms; and
m and p are integers from 0 to 4, wherein the sum total of m and p is 4.

29. The process of claim 28, wherein the ZrX₄ or Zr(XO₄)₄ compound is coordinated with another ligand selected from the group consisting of tetrahydrofuran, N,N-dimethylformamide, and water.

30. The process of claim 28, wherein the zirconium(IV) metal salt is complexed with another metal salt selected from the group consisting of iron(III), tin(IV), gallium(III), and hafnium(IV).

31. The process of claim 21, wherein the zirconium(IV) metal salt is conjugated to a solid support.

32. The process of claim 31, wherein the solid support is selected from the group consisting of silicas, alumina, titania, carbondium, zirconia, activated charcoal, zeolites, clays, polymers, ceramics, activated carbon, porous metal supports, and a combination thereof.

33. The process of claim 21, wherein the concentration of the zirconium(IV) metal salt is from about 1% to about 20% by weight of the lipid material.

34. The process of claim 21, wherein the N-heterocyclic carbene is a compound having a formula selected from the group consisting of (VI) and (VII): 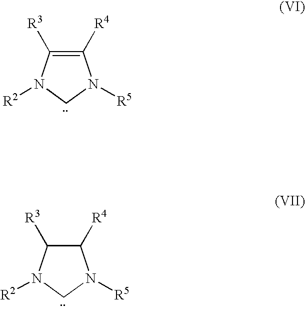
wherein:
R² and R⁵ are independently selected from the group consisting of a hydrocarbyl group having from one carbon atom to about twelve carbon atoms and a substituted hydrocarbyl group having from one carbon atom to about twelve carbon atoms; and
R³ and R⁴ are independently selected from the group consisting of a hydrogen atom, a halogen atom, and a hydrocarbyl group having from one carbon atom to about six carbon atoms.

35. The process of claim 34, wherein the N-heterocyclic carbene is selected from the group consisting of 1,3-bis(2,4,6-trimethylphenyl)imidazol-2-ylidene, 1,3-bis(2,6-diisopropylphenyl)imidazol-2-ylidene, 1,3-diisopropylimidazol-2-ylidene, 1,3-bis(1-adamantyl)imidazol-2-ylidene, 1,3-dicyclohexylimidazol-2-ylidene, 1-butyl-3-methylimidzol-2-ylidene, 1,3-diisopropylimidzolin-2-ylidene, a metal derivative, a dimer thereof, and a combination thereof.

36. The process of claim 21, wherein the concentration of the N-heterocyclic carbene is about 0.02% to about 40% by weight of the lipid material.

37. The process of claim 21, wherein the N-heterocyclic carbene is conjugated to a solid support.

38. The process of claim 37, wherein the solid support is selected from the group consisting of silicas, alumina, titania, carbondium, zirconia, activated charcoal, zeolites, clays, polymers, ceramics, activated carbon, porous metal supports, and a combination thereof.

39. The process of claim 21, wherein the alkaline agent is selected from the group consisting of sodium hydroxide, potassium hydroxide, sodium methoxide₁ and potassium methoxide.

40. The process of claim 21, wherein the process is conducted in a mode selected from the group consisting of batch, semi-continuous, and continuous.

41. The process of claim 21, wherein the lipid material is soybean oil, the alcohol is methanol, the first catalyst is zirconium tetrachloride conjugated to alumina, and the second catalyst is 1-butyl-3-methylimidzol-2-ylidene.

42. The process of claim 21, wherein the yield of fatty acid alkyl esters is at least about 90%.

43. The process of claim 21, further comprising post-treating the biodiesel solution to remove reaction byproducts and/or impurities.
